(19) United States
(12) Patent Application Publication
(10) Pub. No.: US 2009/0232863 A1
(43) Pub. Date: Sep. 17, 2009

(54) Biodegradable Carbon Diazeniumdiolate Based Nitric Oxide Donating Polymers

(75) Inventors: Peiwen Cheng, Santa Rosa, CA (US); Kishore Udipi, Santa Rosa, CA (US); Mingfei Chen, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/049,618

(22) Filed: Mar. 17, 2008

(51) Int. Cl. A61K 33/00; A61F 2/82; A61F 2/00

(52) U.S. Cl. 424/422; 424/718

(57) ABSTRACT

Disclosed herein are implantable medical devices coated with or comprising bioabsorbable carbon-based nitric oxide-donating polymers that upon exposure to physiological environments donate nitric oxide (NO).

### FIELD OF THE INVENTION

The present disclosure relates to nitric oxide (NO) donating polymers for coating and manufacturing medical devices.

### BACKGROUND OF THE INVENTION

Nitric oxide (NO) is a simple diatomic molecule that plays a diverse and complex role in cellular physiology. Less than 25 years ago NO was primarily considered a smog component formed during the combustion of fossil fuels mixed with air. However, as a result of the pioneering work of Ferid Murad et al. it is now known that NO is a powerful signaling compound and cytotoxic/cytostatic agent found in nearly every tissue including endothelial cells, neural cells and macrophages. Mammalian cells synthesize NO using a two step enzymatic process that oxidizes L-arginine to N-ω-hydroxy-L-arginine, which is then converted into L-citrulline and an uncharged NO free radical. Three different nitric oxide synthase enzymes regulate NO production. Neuronal nitric oxide synthase (NOSI, or nNOS) is formed within neuronal tissue and plays an essential role in neurotransmission; endothelial nitric oxide synthase (NOS3 or eNOS), is secreted by endothelial cells and induces vasodilatation; inducible nitric oxide synthase (NOS2 or iNOS) is principally found in macrophages, hepatocytes and chondrocytes and is associated with immune cytotoxicity.

Neuronal NOS and eNOS are constitutive enzymes that regulate the rapid, short-term release of small amounts of NO. These minute amounts NO activate guanylate cyclase which elevates cyclic guanosine monophosphate (cGMP) concentrations which in turn increase intracellular Ca²⁺ levels. Increased intracellular Ca²⁺ concentrations result in smooth muscle relaxation which accounts for the vasodilating effects of NO. Inducible NOS is responsible for the sustained release of larger amounts of NO and is activated by extracellular factors including endotoxins and cytokines. These higher NO levels play a key role in cellular immunity.

Medical research, especially in the fields of vascular surgery and interventional cardiology, is rapidly discovering therapeutic applications for NO. Procedures used to clear blocked arteries such as percutaneous transluminal coronary angioplasty (PTCA) (also known as balloon angioplasty) and atherectomy and/or stent placement can result in vessel wall injury at the site of balloon expansion or stent deployment. In response to this injury a complex multi-factorial process known as restenosis can occur whereby the previously opened vessel lumen narrows and becomes re-occluded. Restenosis is initiated when thrombocytes (platelets) migrating to the injury site release mitogens into the injured endothelium. Thrombocytes begin to aggregate and adhere to the injury site initiating thrombogenesis, or clot formation. As a result, the previously opened lumen begins to narrow as thrombocytes and fibrin collect on the vessel wall. In a more frequently encountered mechanism of restenosis, the mitogens secreted by activated thrombocytes adhering to the vessel wall stimulate overproliferation of vascular smooth muscle cells during the healing process, restricting or occluding the injured vessel lumen. The resulting neointimal hyperplasia is the major cause of a stent restenosis.

Recently, NO has been shown to significantly reduce thrombocyte aggregation and adhesion; this combined with NO's direct cytotoxic/cytostatic properties may significantly reduce vascular smooth muscle cell proliferation and help prevent restenosis. Thrombocyte aggregation occurs within minutes following the initial vascular insult and once the cascade of events leading to restenosis is initiated, irreparable damage can result. Moreover, the risk of thrombogenesis and restenosis persists until the endothelium lining the vessel lumen has been repaired. Therefore, it is essential that NO, or any anti-restenotic agent, reach the injury site immediately.

One approach for providing a therapeutic level of NO at an injury site is to increase systemic NO levels prophylactically. This can be accomplished by stimulating endogenous NO production or using exogenous NO sources. Methods to regulate endogenous NO release have primarily focused on activation of synthetic pathways using excess amounts of NO precursors like L-arginine, or increasing expression of nitric oxide synthase (NOS) using gene therapy. U.S. Pat. Nos. 5,945,452, 5,891,459 and 5,428,070 describe sustained NO elevation using orally administrated L-arginine and/or L-lysine. However, these methods have not been proven effective in preventing restenosis. Regulating endogenously expressed NO using gene therapy techniques remains highly experimental and has not yet proven safe and effective. U.S. Pat. Nos. 5,268,465, 5,468,630 and 5,658,565, describe various gene therapy approaches.

Exogenous NO sources such as pure NO gas are highly toxic, short-lived and relatively insoluble in physiological fluids. Consequently, systemic exogenous NO delivery is generally accomplished using organic nitrate prodrugs such as nitroglycerin tablets, intravenous suspensions, sprays and transdermal patches. The human body rapidly converts nitroglycerin into NO; however, enzyme levels and co-factors required to activate the prodrug are rapidly depleted, resulting in drug tolerance. Moreover, systemic NO administration can have devastating side effects including hypotension and free radical cell damage. Therefore, using organic nitrate prodrugs to maintain systemic anti-restenotic therapeutic blood levels is not currently possible.

Therefore, considerable attention has been focused on localized, or site specific, NO delivery to ameliorate the disadvantages associated with systemic prophylaxis. Implantable medical devices and/or local gene therapy techniques including medical devices coated with NO-releasing compounds, or vectors that deliver NOS genes to target cells, have been evaluated. Like their systemic counterparts, gene therapy techniques for the localized NO delivery have not been proven safe and effective. There are still significant technical hurdles and safety concerns that must be overcome before site-specific NOS gene delivery will become a reality.

However, significant progress has been made in the field of localized exogenous NO application. To be effective at preventing restenosis an inhibitory therapeutic such as NO must be administered for a sustained period at therapeutic levels. Consequently, any NO-releasing medical device used to treat restenosis must be suitable for implantation. An ideal candidate device is the vascular stent. Therefore, a stent that safely provides therapeutically effective amounts of NO to a precise location would represent a significant advance in restenosis treatment and prevention.

Nitric Oxide releasing compounds suitable for in vivo applications have been developed by a number of investigators. As early as 1960 it was demonstrated that nitric oxide gas could be reacted with amines, for example, diethylamine, to form NO-releasing anions having the following general formula R—R′N—N(O)NO. Salts of these compounds could spontaneously decompose and release NO in solution.

Nitric Oxide releasing compounds with sufficient stability at body temperatures to be useful as therapeutics were ultimately developed by Keefer et al. as described in U.S. Pat. Nos. 4,954,526, 5,039,705, 5,155,137, 5,212,204, 5,250,550, 5,366,997, 5,405,919, 5,525,357 and 5,650,447 all of which are herein incorporated by reference.

The in vivo half-life of NO, however, is limited, causing difficulties in delivering NO to the intended area. Therefore NO-releasing compounds which can produce extended release of NO are needed. Several exemplary NO-releasing compounds have been developed for this purpose, including for example a NO donating aspirin derivative, amyl nitrite and isosorbide dinitrate. Additionally, biocompatible polymers having NO adducts (see, for example, U.S. Patent Publications 2006/0008529 and 2004/0037836) that release NO in a controlled manner have been reported.

Secondary amines have the ability to bind two NO molecules and release them in an aqueous environment. The general structure of an exemplary secondary amine capable of binding two NO molecules is depicted below in Formula 1, referred to hereinafter a diazeniumdiolate, (wherein M is a counterion, and can be a metal, with the appropriate charge, or a proton and wherein R is a generic notation for organic and inorganic chemical groups). Exposing secondary amines to basic conditions while incorporating NO gas under high pressure leads to the formation of nitrogen-based diazeniumdiolates.

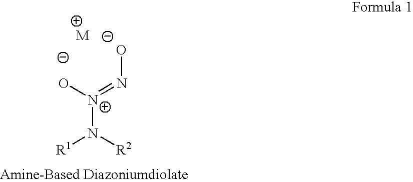

However, nitrogen-based diazeniumdiolate-containing polymers cannot be formulated as bioabsorbable polymers due to the possible breakdown of the nitrogen-based diazeniumdiolate moiety into nitrosamines which are carcinogens and irritants. Therefore bioabsorbable NO-donating polymers that do not incorporate nitrogen-based diazeniumdiolates are needed. Described herein are carbon-based NO-donating polymers.

### SUMMARY OF THE INVENTION

The present description relates to bioabsorbable carbon based diazeniumdiolate (C-based) nitric oxide (NO) donating polymers suitable for forming and coating medical devices. The polymers can have polyester and polyether backbones and are comprised of monomers including, but not limited to, ε-caprolactone, polyethylene glycol (PEG), trimethylene carbonate, lactide, glycolide and their derivatives. Structural integrity and mechanical durability are provided through the use of lactide and glycolide. Elasticity is provided by caprolactone and trimethylene carbonate. Varying the monomer ratios allows the practitioner to fine tune, or modify, the properties of the C-based NO releasing polymer to control physical properties. The polymers contain acidic carbon bonded hydrogens that upon treatment with base are de-protonated, enabling the resulting carbanion to react with individual NO molecules producing C-based diazeniumdiolates. The polymers can also be suitable for manufacturing implantable medical devices. In one embodiment, a medical device is manufactured from a bioabsorbable biocompatible polymer. In another embodiment, the bioabsorbable biocompatible polymer is provided as a coating on a medical device. In yet another embodiment, a drug is provided in the bioabsorbable biocompatible polymer medical device or coating.

A medical device is described herein comprising: a nitric oxide (NO)-releasing, biocompatible, biodegradable polymer having the general structure of formula 7:

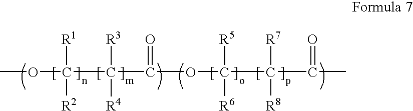

wherein m is 0 or 1; n is 0 to 10; p is 0 or 1; o is 0 to 10, R¹, R², R⁵, R⁶ is each individually hydrogen, a C₁₋₆ alkyl group, a diazeniumdiolate, if m or p is 0. R³, R⁴, R⁷ R⁸ is individually hydrogen or a diazeniumdiolate, if m or p is 1; and wherein at least one of R¹⁻⁸ must be a diazeniumdiolate. In one embodiment, the polymer comprises monomers selected from the group consisting of ε-caprolactone, trimethylene carbonate, 2-acetylbutyrolactone, Formula 10, 4-tert-butyl caprolactone, N-acetyl caprolactone, cyclohexyl caprolactones, lactide, glycolide, p-dioxanone, β-butyrolactones, γ-butyrolactones, γ-valerolactone, δ-valerolactone and phosphate ester.

In one embodiment, the diazeniumdiolate group is further stabilized by a counterion selected from the group consisting of sodium, potassium, a proton, and lithium.

In one embodiment, the medical device is implantable and is selected from the group consisting of vascular stents, shunts, vascular grafts, stent grafts, heart valves, catheters, pacemakers, pacemaker leads, bile duct stents and defibrillators.

In one embodiment, the polymer further comprises at least one drug that is selected from the group consisting of anti-proliferatives, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides and transforming nucleic acids.

In one embodiment, the biodegradable polymer comprises a compound according to a general structure of Formula 8a:

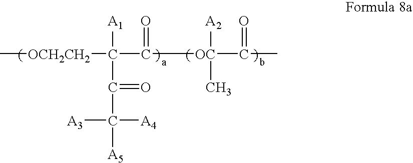

wherein a is an integer from 1 to about 20,000; b is an integer from about 1 to about 100 and the sum of a and b is at least 2; and A¹-A⁵ are individually hydrogen, C₁₋₆ alkyl or a diazeniumdiolate group, and at least one of A¹⁻⁵ must be a diazeniumdiolate.

In another embodiment, the biodegradable polymer comprises a compound according to a general structure of Formula 9a:

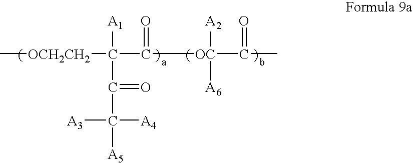

wherein a is an integer from 1 to about 20,000; b is an integer from about 1 to about 100 and the sum of a and b is at least 2; and A¹-A⁶ are individually hydrogen, C₁₋₆ alkyl or a diazeniumdiolate group, and at least one of A¹⁻⁶ must be a diazeniumdiolate.

In one embodiment, the biodegradable polymer comprises a compound according to a general structure of Formula 10a:

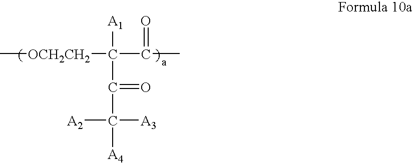

wherein a is an integer from 1 to about 20,000; and A¹-A⁴ are individually hydrogen, C₁₋₆ alkyl or a diazeniumdiolate group, and at least one of A¹⁻⁴ must be a diazeniumdiolate.

In one embodiment, biodegradable polymer comprises a compound according to a general structure of Formula 11a:

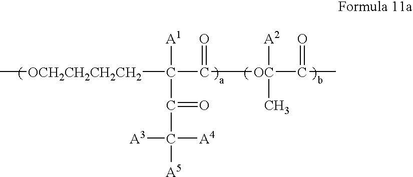

wherein a is an integer from 1 to about 20,000; b is an integer from about 1 to about 100 and the sum of a and b is at least 2; and A¹-A⁵ are individually hydrogen, C₁₋₆ alkyl or a diazeniumdiolate group, and at least one of A¹⁻⁵ must be a diazeniumdiolate.

In one embodiment, the biodegradable polymer comprises a compound according to a general structure of Formula 12a:

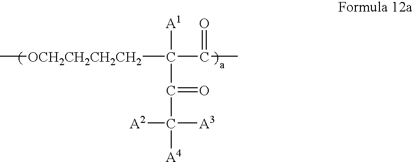

wherein a is an integer from 1 to about 20,000; and A¹-A⁴ are individually hydrogen, C₁₋₆ alkyl or a diazeniumdiolate group, and at least one of A¹⁻⁴ must be a diazeniumdiolate.

In one embodiment, the biodegradable polymer comprises a compound according to a general structure of Formula 13a:

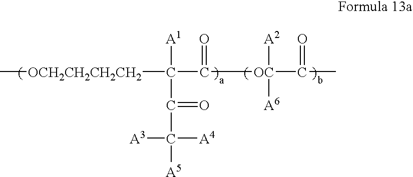

wherein a is an integer from 1 to about 20,000; b is an integer from about 1 to about 100 and the sum of a and b is at least 2; and A¹-A⁶ are individually hydrogen, C₁₋₆ alkyl or a diazeniumdiolate group, and at least one of A¹⁻⁶ must be a diazeniumdiolate.

In one embodiment, a medical device is described comprising: a nitric oxide (NO)-releasing, biocompatible, biodegradable polymer having at least one diazeniumdiolate group bound to a carbon adjacent to a carbonyl group. In another embodiment, the polymer comprises monomers selected from the group consisting of ε-caprolactone, trimethylene carbonate, 2-acetylbutyrolactone, Formula 10, 4-tert-butyl caprolactone, N-acetyl caprolactone, cyclohexyl caprolactones, lactide, glycolide, p-dioxanone, β-butyrolactones, γ-butyrolactones, γ-valerolactone, δ-valerolactone and phosphate ester. In another embodiment, the diazeniumdiolate group is further stabilized by a counterion selected from the group consisting of sodium, potassium, a proton, and lithium.

In one embodiment, the medical device is implantable and is selected from the group consisting of vascular stents, shunts, vascular grafts, stent grafts, heart valves, catheters, pacemakers, pacemaker leads, bile duct stents and defibrillators.

In one embodiment, the polymer further comprises at least one drug that is selected from the group consisting of anti-proliferatives, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides and transforming nucleic acids.

In one embodiment, the biodegradable polymer comprises a compound according to a general structure of Formula 8a:

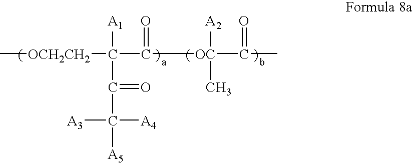

wherein a is an integer from 1 to about 20,000; b is an integer from about 1 to about 100 and the sum of a and b is at least 2; and A¹-A⁵ are individually hydrogen, C₁₋₆ alkyl or a diazeniumdiolate group, and at least one of A¹⁻⁵ must be a diazeniumdiolate.

In one embodiment, the biodegradable polymer comprises a compound according to a general structure of Formula 9a:

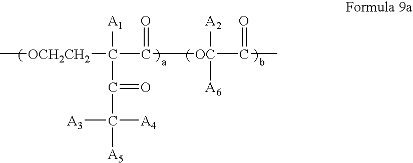

wherein a is an integer from 1 to about 20,000; b is an integer from about 1 to about 100 and the sum of a and b is at least 2; and A¹-A⁶ are individually hydrogen, C₁₋₆ alkyl or a diazeniumdiolate group, and at least one of A¹⁻⁶ must be a diazeniumdiolate.

In one embodiment, the biodegradable polymer comprises a compound according to a general structure of Formula 10a:

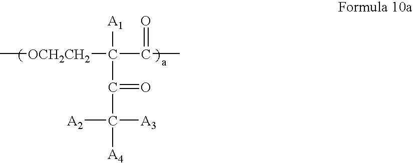

wherein a is an integer from 1 to about 20,000; and A¹-A⁴ are individually hydrogen, C₁₋₆ alkyl or a diazeniumdiolate group, and at least one of A¹⁻⁴ must be a diazeniumdiolate.

In one embodiment, the biodegradable polymer comprises a compound according to a general structure of Formula 11a:

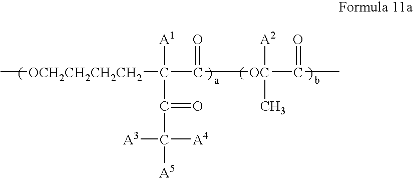

wherein a is an integer from 1 to about 20,000; b is an integer from about 1 to about 100 and the sum of a and b is at least 2; and A¹-A⁵ are individually hydrogen, C₁₋₆ alkyl or a diazeniumdiolate group, and at least one of A¹⁻⁵ must be a diazeniumdiolate.

In one embodiment, the biodegradable polymer comprises a compound according to a general structure of Formula 12a:

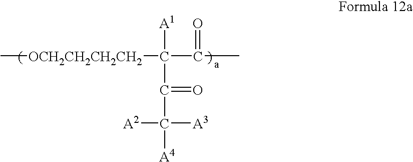

wherein a is an integer from 1 to about 20,000; and A¹-A⁴ are individually hydrogen, C₁₋₆ alkyl or a diazeniumdiolate group, and at least one of A¹⁻⁴ must be a diazeniumdiolate.

In one embodiment, the biodegradable polymer comprises a compound according to a general structure of Formula 13a:

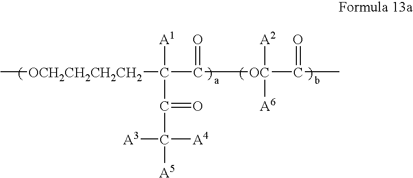

wherein a is an integer from 1 to about 20,000; b is an integer from about 1 to about 100 and the sum of a and b is at least 2; and A¹-A⁶ are individually hydrogen, C₁₋₆ alkyl or a diazeniumdiolate group, and at least one of A¹⁻⁶ must be a diazeniumdiolate.

In one embodiment, a vascular stent is described comprising: comprising a NO-releasing, biocompatible, biodegradable polymer having at least one diazeniumdiolate group bound to a carbon adjacent to a carbonyl group further comprising the general structure of formula 7:

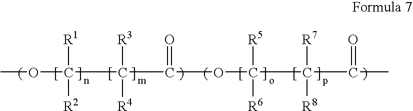

wherein m is 0 or 1; n is 0 to 10; p is 0 or 1; o is 0 to 10, R¹, R², R⁵, R⁶ is each individually hydrogen, a C₁₋₆ alkyl group, a diazeniumdiolate, if m or p is 0. R³, R⁴, R⁷ R⁸ is individually hydrogen or a diazeniumdiolate, if m or p is 1; wherein at least one of R¹⁻⁸ must be a diazeniumdiolate; and wherein said biodegradable polymer further comprises zotarolimus.

### Definition of Terms

Prior to setting forth the invention, it may be helpful to an understanding thereof to set forth definitions of certain terms that will be used hereinafter:

Lactide: As used herein, lactide refers to 3,6-dimethyl-1,4-dioxane-2,5-dione (Formula 2). More commonly lactide is also referred to herein as the heterodimer of R and S forms of lactic acid, the homodimer of the S form of lactic acid and the homodimer of the R form of lactic acid. Lactic acid and lactide are used interchangeably herein. The term dimer is well known to those ordinarily skilled in the art.

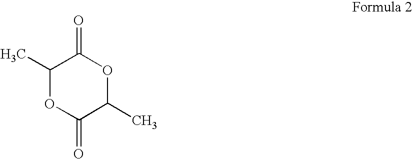

Glycolide: As used herein, glycolide refers to a molecule having the general structure of Formula 3.

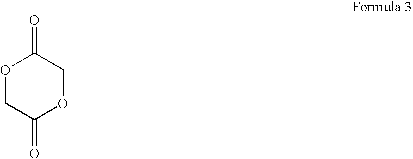

4-tert-butyl caprolactone: As used herein 4-tert-butyl caprolactone refers to a molecule having the general structure of Formula 4.

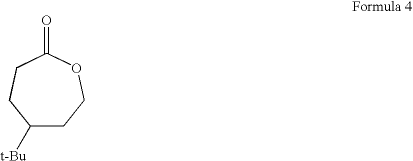

N-acetyl caprolactone: As used herein N-acetyl caprolactone refers to a molecule having the general structure of Formula 5.

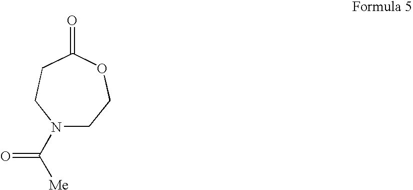

Backbone: As used here in “backbone” refers to the main chain of a polymer or copolymer. A “polyester backbone” as used herein refers to the main chain of a bioabsorbable polymer comprising ester linkages. A “polyether backbone” as used herein refers to the main chain of a bioabsorbable polymer comprising ether linkages. An exemplary polyether is polyethylene glycol (PEG).

Bioabsorbable: As used herein “bioabsorbable” refers to a polymeric composition that is biocompatible and subject to being broken down in vivo through the action of normal biochemical pathways. From time-to-time bioabsorbable and biodegradable may be used interchangeably, however they are not coextensive. Biodegradable polymers may or may not be reabsorbed into surrounding tissues, however all bioabsorbable polymers are considered biodegradable.

Biocompatible: As used herein “biocompatible” shall mean any material that does not cause injury or death to the animal or induce an adverse reaction in an animal when placed in intimate contact with the animal's tissues. Adverse reactions include inflammation, infection, fibrotic tissue formation, cell death, or thrombosis.

Copolymer: As used here in a “copolymer” will be defined as a macromolecule produced by the simultaneous or step-wise polymerization of two or more dissimilar units such as monomers. Copolymer shall include bipolymers (two dissimilar units), terpolymers (three dissimilar units), etc.

Controlled release: As used herein “controlled release” refers to the release of a bioactive compound from a medical device surface at a predetermined rate. Controlled release implies that the bioactive compound does not come off the medical device surface sporadically in an unpredictable fashion and does not “burst” off of the device upon contact with a biological environment (also referred to herein a first order kinetics) unless specifically intended to do so. However, the term “controlled release” as used herein does not preclude a “burst phenomenon” associated with deployment. In some embodiments, an initial burst of drug may be desirable followed by a more gradual release thereafter. The release rate may be steady state (commonly referred to as “timed release” or zero order kinetics), that is the drug is released in even amounts over a predetermined time (with or without an initial burst phase) or may be a gradient release. A gradient release implies that the concentration of drug released from the device surface changes over time.

Drug(s): As used herein “drug” shall include any bioactive agent, pharmaceutical compound or molecule having a therapeutic effect in an animal. Exemplary, non-limiting examples include anti-proliferatives including, but not limited to, macrolide antibiotics including FKBP 12 binding compounds, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides, and transforming nucleic acids. Drugs can also include cytostatic compounds, chemotherapeutic agents, analgesics, statins, nucleic acids, polypeptides, growth factors, and delivery vectors including, but not limited to, recombinant micro-organisms, and liposomes.

Exemplary FKBP 12 binding compounds include sirolimus (rapamycin), tacrolimus (FK506), everolimus (certican or RAD-001), temsirolimus (CCI-779 or amorphous rapamycin 42-ester with 3-hydroxy-2-(hydroxymethyl)-2-methylpropionic acid) and zotarolimus (ABT-578). Additionally, other rapamycin hydroxyesters may be used in combination with the polymers.

Ductility: As used herein “ductility, or ductile” is a polymer attribute characterized by the polymer's resistance to fracture or cracking when folded, stressed or strained at operating temperatures. When used in reference to the polymer coating compositions the normal operating temperature for the coating will be between room temperature and body temperature or approximately between 15° C. and 40° C. Polymer durability in a defined environment is often a function of its elasticity/ductility.

Functional Side Chain: As used herein “functional side chain” encompasses a first chemical constituent(s) typically capable of binding to a second chemical constituent(s), wherein the first chemical constituent modifies a chemical or physical characteristic of the second chemical constituent. Functional groups associated with the functional side chains include vinyl groups, hydroxyl groups, oxo groups, carboxyl groups, thiol groups, amino groups, phosphate groups and others known to those skilled in the art and as depicted in the present specification and claims.

Glass Transition Temperature (T_{g}): As used herein glass transition temperature (T_{g}) refers to a temperature wherein a polymer structurally transitions from a elastic pliable state to a rigid and brittle state.

Hydrophilic: As used herein in reference to the bioactive agent, the term “hydrophilic” refers to a bioactive agent that has solubility in water of more than 200 micrograms per milliliter.

Hydrophobic: As used herein in reference to the bioactive agent the term “hydrophobic” refers to a bioactive agent that has solubility in water of no more than 200 micrograms per milliliter.

Mₙ: As used herein Mₙ refers to number-average molecular weight. Mathematically it is represented by the following formula:

Mₙ=Σᵢ Nᵢ Mᵢ/Σᵢ Nᵢ, wherein the Nᵢ ^{_{the number of moles whose weight is}} Mᵢ.

M_{w}: As used herein M_{w} refers to weight average molecular weight that is the average weight that a given polymer may have. Mathematically it is represented by the following formula:

M_{w}=Σᵢ Nᵢ Mᵢ²/Σᵢ Nᵢ Mᵢ, wherein Nᵢ is the number of molecules whose weight is Mᵢ.

### DETAILED DESCRIPTION OF THE INVENTION

Disclosed herein are bioabsorbable carbon-based (C-based) nitric oxide (NO)-donating polymers suitable for forming and coating medical devices. The polymers can have polyester and polyether backbones and may be comprised of hydrophilic and hydrophobic monomers.

As used herein, the terms “carbon-based” and “C-based” refer to molecules having the general structure of Formula 6 wherein the NO-donating groups, diazeniumdiolates are bound to carbon atoms. The carbon atoms binding the diazeniumdiolate group are the alpha carbons adjacent to carbonyl carbons. The carbonyl groups can be incorporated into the polymer backbone, can exist on a pendant group attached to the polymer backbone or can exist as a product of the polymer synthesis.

The carbonyl group increases the acidity of the alpha carbon(s) to enable the deprotonation and diazeniumdiolation. In Formula 6, M may be a counter ion including, but not limited to, lithium, sodium, potassium and a proton; the carbon is an alpha carbon either on a pendent group or on the polymer backbone itself. The purpose of the counter ion is to stabilize the diazeniumdiolate group.

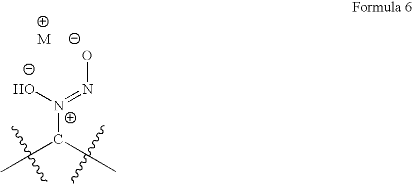

The polymer backbone comprises monomers including, but are not limited to, ε-caprolactone, trimethylene carbonate, lactide, glycolide, p-dioxanone, β-butyrolactones, γ-butyrolactones, γ-valerolactone, δ-valerolactone, phosphate ester, lactones and their derivatives synthesized from cyclic ketones, and their copolymers having anhydride and orthoester segments. Other useful caprolactone monomers include, but are not limited to 4-tert-butyl caprolactone, N-acetyl caprolactone, and cyclohexyl caprolactone.

A generic structure for the diazeniumdiolated polymer is depicted in Formula 7.

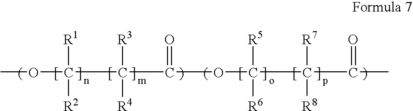

In Formula 7, m is 0 or 1; n is 0 to 10; p is 0 or 1; o is 0 to 10, R¹, R², R⁵, R⁶ is each individually hydrogen, a C₁₋₆ alkyl group, a diazeniumdiolate, if m or p is 0. R³, R⁴, R⁷ R⁸ is individually hydrogen or a diazeniumdiolate, if m or p is 1 and wherein at least one of R¹⁻⁸ must be a diazeniumdiolate;

In order to facilitate the diazeniumdiolation of the polymers, sufficiently acidic protons, such as, but not limited to those of acetyl groups, may be incorporated into the polymers. The functional groups that increase the acidity of the carbon bonded protons in the polymers include, but are not limited to, ketones, sulfones, esters, nitriles, electron withdrawing aryl groups, nitrates, and sulfoxides. The bases used to generate the carbanion include, but are not limited to, potassium methoxide, sodium methoxide, cesium methoxide, lithium methoxide, potassium ethoxide, sodium ethoxide, cesium ethoxide, lithium ethoxide, potassium hydroxide, sodium hydroxide, cesium hydroxide, lithium hydroxide and sodium trimethylsilanolate.

The monomers are either commercially available or synthesized with well known synthetic transformations. For example, cyclohexanone derivatives are treated with peroxides to form lactones (through Baeyer-Villiger oxidation reactions) that are then used as monomers in polymerization reactions. Other cyclic ketones and cyclic ketone derivatives that are used for the syntheses of lactone monomers include, but are not limited to, carbocyclic ketones having 3 to 12 ring carbons, single and multiple substituted carbocyclic ketones having 3 to 12 ring carbons, heterocyclic ketones having 3 to 12 ring carbons, and single and multiple substituted heterocyclic ketones having 3 to 12 ring carbons. The substituents on the rings include, but are not limited to substituted or un-substituted aryl groups having 6 to 12 carbons, hydrocarbons having at least 1 carbon, hydrocarbons bearing acidifying groups, aryl groups bearing acidifying groups, and heteroatom substituted aryl and hydrocarbon substituents. The heteroatoms incorporated in the heterocycles described above include, but are not limited to nitrogen, phosphorus, sulfur, and oxygen.

In one embodiment 2-acetylbutyrolactone is polymerized with lactide in a diol initiated ring-opening polymerization reaction to produce the polymer of Formula 8. These monomers are polymerized, in a non-limiting example, in the presence of a catalyst such as, but not limited to tin(II)-ethylhexanoate, tetrakis Sn (IV) alkoxides, cyclic tin alkoxides, aluminum isopropoxide, zinc lactate, zinc octoate, zinc stearate, zinc salicylate, other organic metallic compounds also used as catalysts such as guanidinium acetate, organolanthanide, enzyme catalysts such as lipase. The diols include, but are not limited to PEG. The polymer represented by Formula 8 can be diazeniumdiolated as described herein.

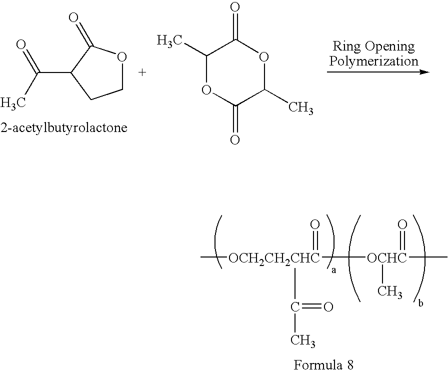

In one embodiment, the a and b units of Formula 8 are individually integers ranging from 1 to 20,000. In additional embodiments, a is an integer ranging from 10 to 20,000; from 50 to 15,000; from 100 to 10,000; from 200 to 5,000; from 500 to 4,000; from 700 to 3,000; or from 1000 to 2000. In additional embodiments, b is an integer ranging from 10 to 20,000; from 50 to 15,000; from 100 to 10,000; from 200 to 5,000; from 500 to 4,000; from 700 to 3,000; or from 1000 to 2000.

The polymer of Formula 8 is diazeniumdiolated to form the polymer of Formula 8a wherein A¹⁻⁵ represent positions on the alpha carbons that can be diazeniumdiolated and wherein a is an integer from 1 to about 20,000; b is an integer from about 1 to about 100 and the sum of a and b is at least 2. At least one of A¹⁻⁵ must be diazeniumdiolated.

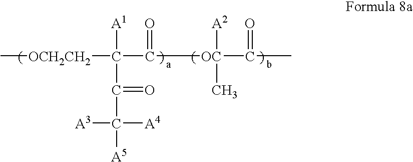

In another embodiment, C-based NO-donating polymers having 2-acetylbutyrolactone and glycolide monomers are produced. An exemplary polymer, produced with these monomers, has the general structure of Formula 9:

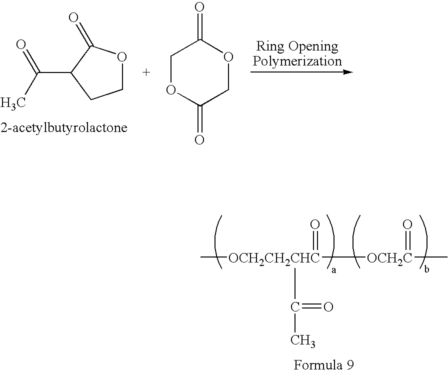

In one embodiment, the a and b units of Formula 9 are integers ranging from 1 to 20,000. In additional embodiments, a is an integer ranging from 10 to 20,000; from 50 to 15,000; from 100 to 10,000; from 200 to 5,000; from 500 to 4,000; from 700 to 3,000; or from 1000 to 2000. In additional embodiments, b is an integer ranging from 10 to 20,000; from 50 to 15,000; from 100 to 10,000; from 200 to 5,000; from 500 to 4,000; from 700 to 3,000; or from 1000 to 2000.

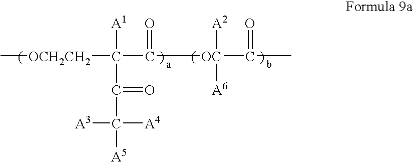

The polymer of Formula 9 is diazeniumdiolated to form the polymer of Formula 9a wherein A¹⁻⁶ represent positions on the alpha carbons that can be diazeniumdiolated and wherein a is an integer from 1 to about 20,000; b is an integer from about 1 to about 100 and the sum of a and b is at least 2. At least one of A¹⁻⁶ must be diazeniumdiolated.

In another embodiment, a C-based NO-donating homopolymer comprising the monomer 2-acetylbutyrolactone is produced. An exemplary polymer produced with 2-acetylbutyrolactone has the general structure of Formula 10:

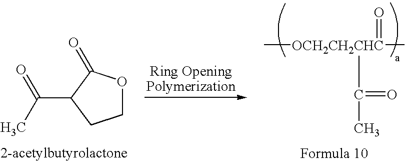

In one embodiment, the a units of Formula 10 are integers ranging from 1 to 20,000. In additional embodiments, a is an integer ranging from 10 to 20,000; from 50 to 15,000; from 100 to 10,000; from 200 to 5,000; from 500 to 4,000; from 700 to 3,000; or from 1000 to 2000.

The polymer of Formula 10 is diazeniumdiolated to form the polymer of Formula 10a wherein A¹⁻⁴ represent positions on the alpha carbon that can be diazeniumdiolated and wherein a is an integer from 1 to about 20,000. At least one of A¹⁻⁴ must be diazeniumdiolated.

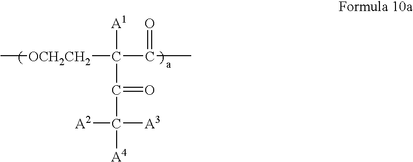

Other acetyl-bearing monomers can be synthesized and, subsequently, polymerized, into the polymers. In one embodiment, Baeyer-Villager reactions are used to produce lactones with ring sizes ranging from 4 to 12 carbons. These lactones are then polymerized through ring-opening polymerization reactions producing C-based NO-donating polymers. In one embodiment a Baeyer-Villager reaction is initiated with 2-acetylcyclohexanone to produce the caprolactone of Formula 10.

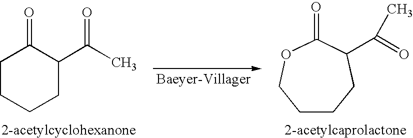

In one embodiment, a C-based NO-donating polymer is synthesized by polymerizing 2-acetylcaprolactone with lactide in a diol-initiated ring-opening polymerization reaction to produce the polymer of Formula 11. These monomers are polymerized in the presence of a catalyst such as tin(II)-ethylhexanoate and a diol such as PEG.

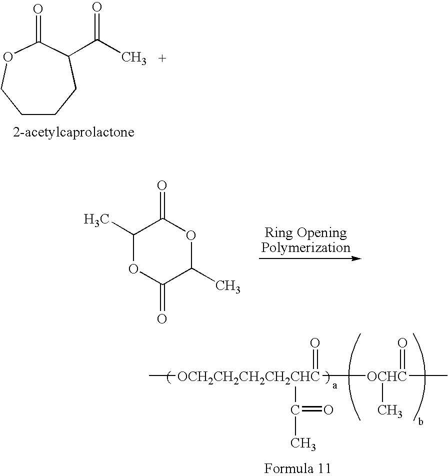

In one embodiment, the a and b units of Formula 11 are integers ranging from 1 to 20,000. In additional embodiments, a is an integer ranging from 10 to 20,000; from 50 to 15,000; from 100 to 10,000; from 200 to 5,000; from 500 to 4,000; from 700 to 3,000; or from 1000 to 2000. In additional embodiments, b is an integer ranging from 10 to 20,000; from 50 to 15,000; from 100 to 10,000; from 200 to 5,000; from 500 to 4,000; from 700 to 3,000; or from 1000 to 2000.

The polymer of Formula 11 is diazeniumdiolated to form the polymer of Formula 11a wherein A¹⁻⁵ represent positions on the alpha carbons that can be diazeniumdiolated and wherein a is an integer from 1 to about 20,000; b is an integer from about 1 to about 100 and the sum of a and b is at least 2. At least one of A¹⁻⁵ must be diazeniumdiolated.

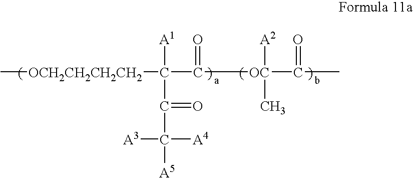

In another embodiment, a C-based NO-donating homopolymer comprising the monomer 2-acetylcaprolactone is produced. An exemplary polymer produced with 2-acetylcaprolactone has the general structure of Formula 12:

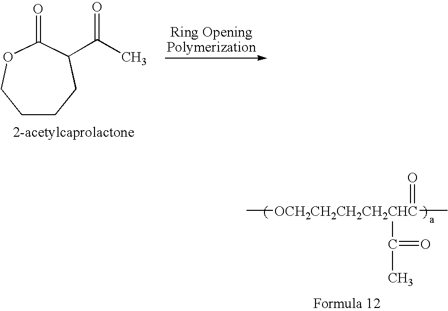

In one embodiment, the a units of Formula 12 are integers ranging from 1 to 20,000. In additional embodiments, a is an integer ranging from 10 to 20,000; from 50 to 15,000; from 100 to 10,000; from 200 to 5,000; from 500 to 4,000; from 700 to 3,000; or from 1000 to 2000.

The polymer of Formula 12 is diazeniumdiolated to form the polymer of Formula 12a wherein A¹⁻⁴ represent positions on the alpha carbon that can be diazeniumdiolated and wherein a is an integer from 1 to about 20,000. At least one of the A¹⁻⁴ must be diazeniumdiolated.

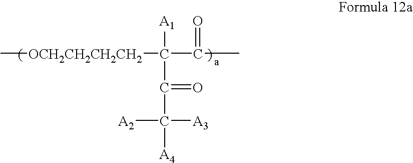

In another embodiment, a C-based NO-donating polymer having monomers comprising 2-acetylcaprolactone and glycolide is produced. An exemplary polymer produced with these monomers has the general structure of Formula 13:

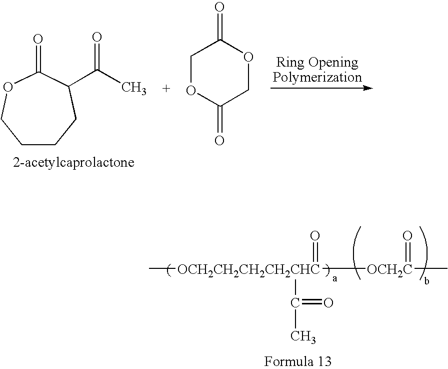

In one embodiment, the a and b units of Formula 13 are integers ranging from 1 to 20,000. In additional embodiments, a is an integer ranging from 10 to 20,000; from 50 to 15,000; from 100 to 10,000; from 200 to 5,000; from 500 to 4,000; from 700 to 3,000; or from 1000 to 2000. In additional embodiments, b is an integer ranging from 10 to 20,000; from 50 to 15,000; from 100 to 10,000; from 200 to 5,000; from 500 to 4,000; from 700 to 3,000; or from 1000 to 2000.

The polymer of Formula 13 is diazeniumdiolated to form the polymer of Formula 13a wherein A¹⁻⁶ represent positions on the alpha carbons that can be diazeniumdiolated and wherein a is an integer from 1 to about 20,000; b is an integer from about 1 to about 100 and the sum of a and b is at least 2. At least one of the A¹⁻⁶ must be diazeniumdiolated.

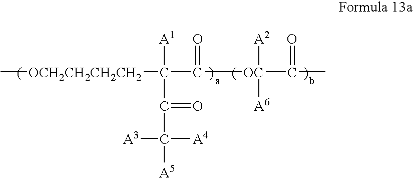

The properties of bioabsorbable C-based NO-donating polymers are a result of the monomers used and the reaction conditions employed in their synthesis including, but not limited to, temperature, solvent choice, reaction time and catalyst choice.

Varying the monomer ratios allows the ordinarily skilled artisan to fine tune, or to modify, the properties of the polymer. The properties of bioabsorbable C-based NO-donating polymers arise from the monomers used and the reaction conditions employed in their synthesis including but not limited to, temperature, solvents, reaction time and catalyst choice.

Fine tuning, or modifying, the glass transition temperature (T_{g}) of the bioabsorbable C-based NO-donating polymers is also taken into account. Drug elution from polymers depends on many factors including density, the drug to be eluted, molecular composition of the polymer and T_{g}. Higher T_{g}s, for example temperatures above 40° C., result in more brittle polymers while lower T_{g}s, e.g. lower than 40° C., result in more pliable and elastic polymers at higher temperatures. Drug elution is slow from polymers that have high T_{g}s while faster rates of drug elution are observed with polymers possessing low T_{g}s. In one embodiment, the T_{g} of the polymer is selected to be lower than 37° C.

In one embodiment, the polymers can be used to form and coat medical devices. Coating polymers having relatively high T_{g}s can result in medical devices with unsuitable drug eluting properties as well as unwanted brittleness. In the cases of polymer-coated vascular stents, a relatively low T_{g} in the coating polymer effects the deployment of the vascular stent. For example, polymer coatings with low T_{g}s are “sticky” and adhere to the balloon used to expand the vascular stent during deployment, causing problems with the deployment of the stent. Low T_{g} polymers, however, have beneficial features in that polymers having low T_{g}s are more elastic at a given temperature than polymers having higher T_{g}s. Expanding and contracting a polymer-coated vascular stent mechanically stresses the coating. If the coating is too brittle, i.e. has a relatively high T_{g}, then fractures may result in the coating possibly rendering the coating inoperable. If the coating is elastic, i.e. has a relatively low T_{g}, then the stresses experienced by the coating are less likely to mechanically alter the structural integrity of the coating. Therefore, the T_{g}s of the polymers can be fine tuned for appropriate coating applications by a combination of monomer composition and synthesis conditions. The polymers are engineered to have adjustable physical properties enabling the practitioner to choose the appropriate polymer for the function desired.

In order to tune, or modify, the polymers, a variety of properties are considered including, but not limited to, T_{g}, connectivity, molecular weight and thermal properties.

The C-based NO-donating polymers donate NO once exposed to a physiological environment. The rates of NO release from the polymers can be fine tuned by selecting the appropriate monomer ratios and diazeniumdiolate positive counterions.

Medical devices, including implantable medical devices, are fabricated and/or coated with the polymers disclosed herein and therefore the physical properties of the polymers are considered in light of the specific application at hand. Physical properties of the polymers can be fine tuned so that the polymers can optimally perform for their intended use. Properties that can be fine tuned, without limitation, include T_{g}, molecular weight (both Mₙ and M_{w}), polydispersity index (PDI, the quotient of M_{w}/Mₙ), degree of elasticity and degree of amphiphlicity. In one embodiment, the T_{g} of the polymers range from about −10° C. to about 85° C. In still another embodiment, the PDI of the polymers range from about 1.35 to about 4. In another embodiment, the T_{g} of the polymers ranges form about 0° C. to about 40° C. In still another embodiment, the PDI of the polymers range from about 1.5 to about 2.5.

Implantable medical devices suitable for coating with the C-based NO-donating polymers include, but are not limited to, vascular stents, stent grafts, urethral stents, bile duct stents, catheters, guide wires, pacemaker leads, bone screws, sutures and prosthetic heart valves. The polymers are suitable for fabricating implantable medical devices. Medical devices which can be manufactured from the C-based NO-donating polymers include, but are not limited to, vascular stents, stent grafts, urethral stents, bile duct stents, catheters, guide wires, pacemaker leads, bone screws, sutures and prosthetic heart valves.

The polymeric coatings are intended for medical devices deployed in a hemodynamic environment and possess excellent adhesive properties. That is, the coating must be stably linked to the medical device surface. Many different materials can be used to fabricate the implantable medical devices including, but not limited to, stainless steel, nitinol, aluminum, chromium, titanium, gold, cobalt, ceramics, and a wide range of synthetic polymeric and natural materials including, but not limited to, collagen, fibrin and plant fibers. All of these materials, and others, may be used with the polymeric coatings described herein. Furthermore, the polymers can be used to fabricate an entire medical device.

There are many theories that attempt to explain, or contribute to our understanding of how polymers adhere to surfaces. The most important forces include electrostatic and hydrogen bonding. However, other factors including wettability, absorption and resiliency also determine how well a polymer will adhere to different surfaces. Therefore, polymer base coats, or primers are often used in order to create a more uniform coating surface.

The C-based NO donating polymeric coatings can be applied to medical device surfaces, either primed or bare, in any manner known to those skilled in the art. Applications methods include, but are not limited to, spraying, dipping, brushing, vacuum-deposition, electrostatic spray coating, plasma coating, spin coating electrochemical coating, and others.

Moreover, the C-based NO-donating polymeric coatings may be used with a cap coat. A cap coat as used herein refers to the outermost coating layer applied over another coating. A C-based NO-donating polymer coating is applied over the primer coat. Then, a polymer cap coat is applied over the NO donating polymeric coating. The cap coat may optionally serve as a diffusion barrier to control NO release. The cap coat may be merely a biocompatible polymer applied to the surface of the sent to protect the stent and have no effect on NO release rates.

The C-based NO-donating polymers are also useful for the delivery and controlled release of drugs. Drugs that are suitable for release from the polymers include, but are not limited to, anti-proliferative compounds, cytostatic compounds, toxic compounds, anti-inflammatory compounds, chemotherapeutic agents, analgesics, antibiotics, protease inhibitors, statins, nucleic acids, polypeptides, growth factors and delivery vectors including recombinant micro-organisms, liposomes, and the like.

In one embodiment, the drugs controllably released include, but are not limited to, macrolide antibiotics including FKBP-12 binding agents. Exemplary drugs of this class include sirolimus (rapamycin), tacrolimus (FK506), everolimus (certican or RAD-001), temsirolimus (CCI-779 or amorphous rapamycin 42-ester with 3-hydroxy-2-(hydroxymethyl)-2-methylpropionic acid as disclosed in U.S. patent application Ser. No. 10/930,487) and zotarolimus (ABT-578; see U.S. Pat. Nos. 6,015,815 and 6,329,386). Additionally, other rapamycin hydroxyesters as disclosed in U.S. Pat. No. 5,362,718 may be used in combination with the polymers. The entire contents of all of preceding patents and patent applications are herein incorporated by reference for all they teach related to FKBP-12 binding compounds and the derivatives.

### EXAMPLES

The following non limiting examples provide methods for the synthesis of exemplary polymers according to the teachings of the present invention.

### Example 1

### Synthesis of a Polymer of Formula 8

To a reaction vessel is added polyethylene glycol (PEG) with molecular weight of about 3500 (1.3 g, about 0.4 mmol), 2-acetylbutyrolactone (19 g, 150 mmol), dl lactide (35 g, 243 mmol) and tin(II)2-ethylhexanoate (0.05 g, 0.1 mmol). The vessel is purged with nitrogen gas. The mixture is heated (150° C.) and stirred (320 rpm) for 24 hours then cooled to ambient temperature. The polymer is discharged and dissolved in chloroform (2000 mL). Methanol (500 mL) is added precipitating the polymer from solution. The solution is filtered and the mother liquor disregarded. The solid polymers are then re-dissolved in chloroform and poured into Teflon trays.

### Example 2

### Synthesis of a Polymer of Formula 8a

Polymers dissolved (typically 10 mg/50 ml) in THF are placed in a high pressure reaction vessel. An inert gas (including, but not limited to, argon and nitrogen) is then purged through the vessel. A base dissolved in a solvent (typically sodium methoxide or potassium methoxide in methanol) are then added in excess (typically 110% to 200%). The reaction is allowed to stir and the vessel purged with NO gas. The pressure of NO gas is increased (typically at least 15 psi) and the reaction mixture is then stirred further for at least 24 hours. At the end of the required time for the formation of diazeniumdiolates, dry hydrophobic solvents (typically hexanes or methyl tert-butyl ether) are added to aid in the precipitation of the polymers. The polymers are then filtered and dried.

### Example 3

### Formation of Diazeniumdiolates

Polymers dissolved (typically 10 mg/50 mL) in THF are placed in a high pressure reaction vessel. At this step, one or more bioactive agents may be included in the polymer solution. An inert gas (including, but not limited to, argon and nitrogen) is then purged through the vessel. A base dissolved in a solvent (typically sodium methoxide or potassium methoxide in methanol) are then added in excess (typically 110% to 200%). The reaction is allowed to stir and the vessel purged with NO gas. The pressure of NO gas is increased (typically at least 15 psi) and the reaction mixture is then stirred further for at least 24 hours. At the end of the required time for the formation of diazeniumdiolates, dry hydrophobic solvents (typically hexanes or methyl tert-butyl ether) are added to aid in the precipitation of the polymers. The polymers are then filtered and dried.

### Example 4

### Manufacturing Implantable Vascular Stents

For exemplary, non-limiting, purposes a vascular stent will be described. A bioabsorbable NO-donating polymer is heated until molten in the barrel of an injection molding machine and forced into a stent mold under pressure. After the molded polymer (which now resembles and is a stent) is cooled and solidified the stent is removed from the mold. In one embodiment the stent is a tubular shaped member having first and second ends and a walled surface disposed between the first and second ends. The walls are composed of extruded polymer monofilaments woven into a braid-like embodiment. In the second embodiment, the stent is injection molded or extruded. Fenestrations are molded, laser cut, die cut, or machined in the wall of the tube. In the braided stent embodiment monofilaments are fabricated from polymer materials that have been pelletized then dried. The dried polymer pellets are then extruded forming a coarse monofilament which is quenched. The extruded and quenched, crude monofilament is then drawn into a final monofilament with an average diameter from approximately 0.01 mm to 0.6 mm, preferably between approximately 0.05 mm and 0.15 mm. Approximately 10 to 50 of the final monofilaments are then woven in a plaited fashion with a braid angle about 90 to 170 degrees on a braid mandrel sized appropriately for the application. The plaited stent is then removed from the braid mandrel and disposed onto an annealing mandrel having an outer diameter of equal to or less than the braid mandrel diameter and annealed at a temperature between about the polymer glass transition temperature and the melting temperature of the polymer blend for a time period between about five minutes and about 18 hours in air, an inert atmosphere or under vacuum. The stent is then allowed to cool and is then cut.

### Example 5

### Coating Implantable Vascular Stents

A 1% solution of a bioabsorbable NO-donating polymer (such as from Example 2) and optionally a bioactive agent such as ABT-578 (in one embodiment in a polymer:drug ratio of 70:30 by weight), in chloroform is sprayed on a vascular stent and allowed to dry producing a controlled release coating on the vascular stent. Next the solubilized polymer (with or without added bioactive agents) is applied to the surfaces of an implantable medical device using methods known to those skilled in the art such as, but not limited to, rolling, dipping, spraying and painting. Excess polymer is removed under a gentle stream of warm inert gas such as, but not limited to argon or dry nitrogen. The release of drug from the stent into a solvent is measured by high performance liquid chromatography (HPLC).

### Example 6

### Formation of Diazeniumdiolates on Polymer-Coated Vascular Stents

A vascular stent coated with at least one polymer from Example 1 is placed in a 13 mm×100 mm glass test tube. Ten milliliters of 3% sodium methoxide in methanol or acetonitrile is added to the test tube, which is then placed in a 250 mL stainless steel Parr® apparatus. The apparatus is degassed by repeated cycles (×10) of pressurization/depressurization with nitrogen gas at 10 atmospheres. Next, the vessel undergoes 2 cycles of pressurization/depressurization with NO at 30 atmospheres. Finally, the vessel is filled with NO at 30 atmospheres and left at room temperature for 24 hrs. After 24 hrs, the vessel is purged of NO and pressurized/depressurized with repeated cycles (×10) of nitrogen gas at 10 atmospheres. The test tube is removed from the vessel and the 3% sodium methoxide solution is decanted. The stent is then washed with 10 mL of methanol (×1) and 10 mL of diethyl ether (×3). The stent is then removed from the test tube and dried under a stream of nitrogen gas. This procedure results in a NO-donating polymer-coated vascular stent.

For exemplary, non-limiting, purposes a vascular stent will be described. A bioabsorbable C-based NO-donating polymer is heated until molten in the barrel of an injection molding machine and forced into a stent mold under pressure. After the molded polymer (which now resembles and is a stent) is cooled and solidified the stent is removed from the mold. In one embodiment the stent is a tubular shaped member having first and second ends and a walled surface disposed between the first and second ends. The walls are composed of extruded polymer monofilaments woven into a braid-like embodiment. In the second embodiment, the stent is injection molded or extruded. Fenestrations are molded, laser cut, die cut, or machined in the wall of the tube. In the braided stent embodiment monofilaments are fabricated from polymer materials that have been pelletized then dried. The dried polymer pellets are then extruded forming a coarse monofilament which is quenched. The extruded and quenched, crude monofilament is then drawn into a final monofilament with an average diameter from approximately 0.01 mm to 0.6 mm, preferably between approximately 0.05 mm and 0.15 mm. Approximately 10 to 50 of the final monofilaments are then woven in a plaited fashion with a braid angle about 90 to 170 degrees on a braid mandrel sized appropriately for the application. The plaited stent is then removed from the braid mandrel and disposed onto an annealing mandrel having an outer diameter of equal to or less than the braid mandrel diameter and annealed at a temperature between about the polymer glass transition temperature and the melting temperature of the polymer blend for a time period between about five minutes and about 18 hours in air, an inert atmosphere or under vacuum. The stent is then allowed to cool and is then cut.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on those embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are individually incorporated by reference herein in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A medical device comprising:
a nitric oxide (NO)-releasing, biocompatible, biodegradable polymer having the general structure of formula 7: 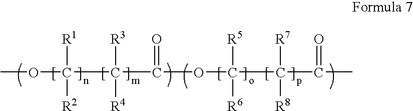
wherein m is 0 or 1; n is 0 to 10; p is 0 or 1; o is 0 to 10, R¹, R², R⁵, R⁶ is each individually hydrogen, a C₁₋₆ alkyl group, a diazeniumdiolate, if m or p is 0, R³, R⁴, R⁷ R⁸ is individually hydrogen or a diazeniumdiolate, if m or p is 1; and wherein at least one of R¹⁻⁸ must be a diazeniumdiolate.

2. The polymer according to claim 1 wherein said polymer comprises monomers selected from the group consisting of ε-caprolactone, trimethylene carbonate, 2-acetylbutyrolactone, Formula 10, 4-tert-butyl caprolactone, N-acetyl caprolactone, cyclohexyl caprolactones, lactide, glycolide, p-dioxanone, β-butyrolactones, γ-butyrolactones, γ-valerolactone, δ-valerolactone and phosphate ester.

3. The polymer according to claim 1 wherein the diazeniumdiolate group is further stabilized by a counterion selected from the group consisting of sodium, potassium, a proton, and lithium.

4. The medical device according to claim 1 wherein said medical device is implantable and is selected from the group consisting of vascular stents, shunts, vascular grafts, stent grafts, heart valves, catheters, pacemakers, pacemaker leads, bile duct stents and defibrillators.

5. The medical device according to claim 1 wherein said polymer further comprises at least one drug that is selected from the group consisting of anti-proliferatives, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides and transforming nucleic acids.

6. The medical device according to claim 1 wherein the biodegradable polymer comprises a compound according to a general structure of Formula 8a: 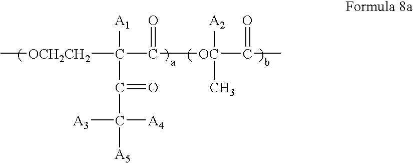
wherein a is an integer from 1 to about 20,000; b is an integer from about 1 to about 100 and the sum of a and b is at least 2; and A¹-A⁵ are individually hydrogen, C₁₋₆ alkyl or a diazeniumdiolate group, and at least one of A¹⁻⁵ must be a diazeniumdiolate.

7. The medical device according to claim 1 wherein the biodegradable polymer comprises a compound according to a general structure of Formula 9a: 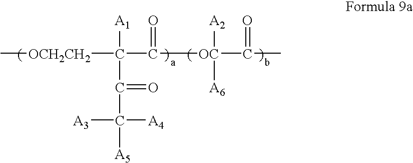
wherein a is an integer from 1 to about 20,000; b is an integer from about 1 to about 100 and the sum of a and b is at least 2; and A¹-A⁶ are individually hydrogen, C₁₋₆ alkyl or a diazeniumdiolate group, and at least one of A¹⁻⁶ must be a diazeniumdiolate.

8. The medical device according to claim 1 wherein the biodegradable polymer comprises a compound according to a general structure of Formula 10a: 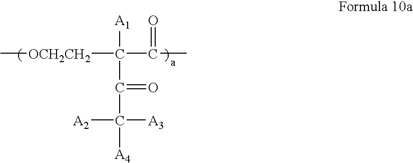
wherein a is an integer from 1 to about 20,000; and A¹-A⁴ are individually hydrogen, C₁₋₆ alkyl or a diazeniumdiolate group, and at least one of A¹⁻⁴ must be a diazeniumdiolate.

9. The medical device according to claim 1 wherein the biodegradable polymer comprises a compound according to a general structure of Formula 11a: 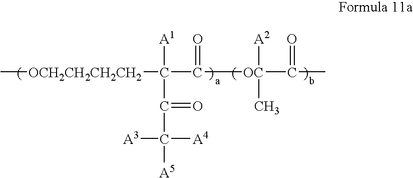
wherein a is an integer from 1 to about 20,000; b is an integer from about 1 to about 100 and the sum of a and b is at least 2; and A¹-A⁵ are individually hydrogen, C₁₋₆ alkyl or a diazeniumdiolate group, and at least one of A¹⁻⁵ must be a diazeniumdiolate.

10. The medical device according to claim 1 wherein the biodegradable polymer comprises a compound according to a general structure of Formula 12a: 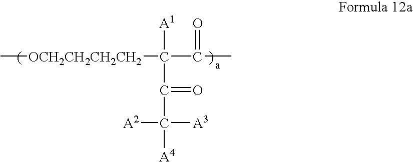
wherein a is an integer from 1 to about 20,000; and A¹-A⁴ are individually hydrogen, C₁₋₆ alkyl or a diazeniumdiolate group, and at least one of A¹⁻⁴ must be a diazeniumdiolate.

11. The medical device according to claim 1 wherein the biodegradable polymer comprises a compound according to a general structure of Formula 13a: 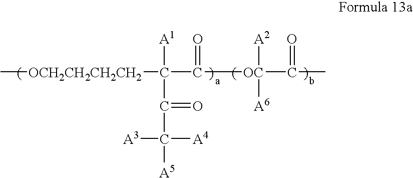
wherein a is an integer from 1 to about 20,000; b is an integer from about 1 to about 100 and the sum of a and b is at least 2; and A¹-A⁶ are individually hydrogen, C₁₋₆ alkyl or a diazeniumdiolate group, and at least one of A¹⁻⁶ must be a diazeniumdiolate.

12. A medical device comprising:
a nitric oxide (NO)-releasing, biocompatible, biodegradable polymer having at least one diazeniumdiolate group bound to a carbon adjacent to a carbonyl group.

13. The polymer according to claim 1 wherein said polymer comprises monomers selected from the group consisting of ε-caprolactone, trimethylene carbonate, 2-acetylbutyrolactone, Formula 10, 4-tert-butyl caprolactone, N-acetyl caprolactone, cyclohexyl caprolactones, lactide, glycolide, p-dioxanone, β-butyrolactones, γ-butyrolactones, γ-valerolactone, δ-valerolactone and phosphate ester.

14. The polymer according to claim 1 wherein the diazeniumdiolate group is further stabilized by a counterion selected from the group consisting of sodium, potassium, a proton, and lithium.

15. The medical device according to claim 1 wherein said medical device is implantable and is selected from the group consisting of vascular stents, shunts, vascular grafts, stent grafts, heart valves, catheters, pacemakers, pacemaker leads, bile duct stents and defibrillators.

16. The medical device according to claim 1 wherein said polymer further comprises at least one drug that is selected from the group consisting of anti-proliferatives, estrogens, chaperone inhibitors, protease inhibitors, protein-tyrosine kinase inhibitors, leptomycin B, peroxisome proliferator-activated receptor gamma ligands (PPARγ), hypothemycin, nitric oxide, bisphosphonates, epidermal growth factor inhibitors, antibodies, proteasome inhibitors, antibiotics, anti-inflammatories, anti-sense nucleotides and transforming nucleic acids.

17. The medical device according to claim 1 wherein the biodegradable polymer comprises a compound according to a general structure of Formula 8a: 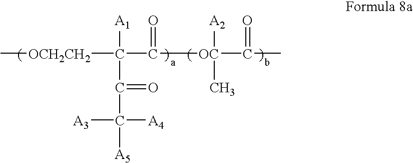
wherein a is an integer from 1 to about 20,000; b is an integer from about 1 to about 100 and the sum of a and b is at least 2; and A¹-A⁵ are individually hydrogen, C₁₋₆ alkyl or a diazeniumdiolate group, and at least one of A¹⁻⁵ must be a diazeniumdiolate.

18. The medical device according to claim 1 wherein the biodegradable polymer comprises a compound according to a general structure of Formula 9a: 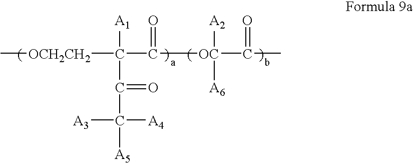
wherein a is an integer from 1 to about 20,000; b is an integer from about 1 to about 100 and the sum of a and b is at least 2; and A¹-A⁶ are individually hydrogen, C₁₋₆ alkyl or a diazeniumdiolate group, and at least one of A¹⁻⁶ must be a diazeniumdiolate.

19. The medical device according to claim 1 wherein the biodegradable polymer comprises a compound according to a general structure of Formula 10a: 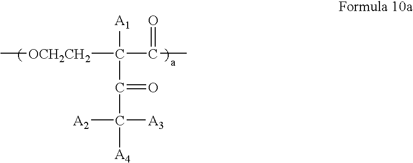
wherein a is an integer from 1 to about 20,000; and A¹-A⁴ are individually hydrogen, C₁₋₆ alkyl or a diazeniumdiolate group, and at least one of A¹⁻⁴ must be a diazeniumdiolate.

20. The medical device according to claim 1 wherein the biodegradable polymer comprises a compound according to a general structure of Formula 11a: 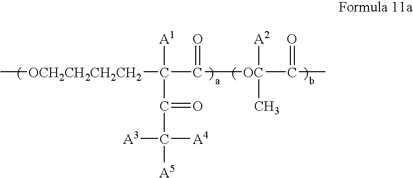
wherein a is an integer from 1 to about 20,000; b is an integer from about 1 to about 100 and the sum of a and b is at least 2; and A¹-A⁵ are individually hydrogen, C₁₋₆ alkyl or a diazeniumdiolate group, and at least one of A¹⁻⁵ must be a diazeniumdiolate.

21. The medical device according to claim 1 wherein the biodegradable polymer comprises a compound according to a general structure of Formula 12a: 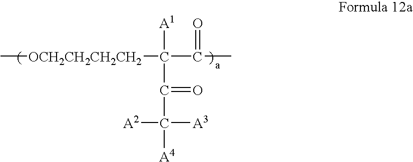
wherein a is an integer from 1 to about 20,000; and A¹-A⁴ are individually hydrogen, C₁₋₆ alkyl or a diazeniumdiolate group, and at least one of A¹⁻⁴ must be a diazeniumdiolate.

22. The medical device according to claim 1 wherein the biodegradable polymer comprises a compound according to a general structure of Formula 13a: 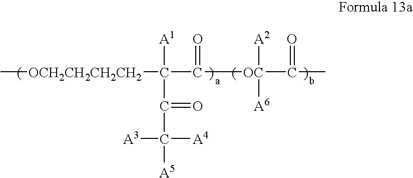
wherein a is an integer from 1 to about 20,000; b is an integer from about 1 to about 100 and the sum of a and b is at least 2; and A¹-A⁶ are individually hydrogen, C₁₋₆ alkyl or a diazeniumdiolate group, and at least one of A¹⁻⁶ must be a diazeniumdiolate.

23. A vascular stent comprising:
a NO-releasing, biocompatible, biodegradable polymer having at least one diazeniumdiolate group bound to a carbon adjacent to a carbonyl group
further comprising the general structure of formula 7: 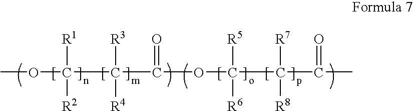
wherein m is 0 or 1; n is 0 to 10; p is 0 or 1; o is 0 to 10, R¹, R², R⁵, R⁶ is each individually hydrogen, a C₁₋₆ alkyl group, a diazeniumdiolate, if m or p is 0. R³, R⁴, R⁷ R⁸ is individually hydrogen or a diazeniumdiolate, if m or p is 1; wherein at least one of R¹⁻⁸ must be a diazeniumdiolate; and
wherein said biodegradable polymer further comprises zotarolimus.
